(19) United States
(12) Patent Application Publication
(10) Pub. No.: US 2005/0255179 A1
(43) Pub. Date: Nov. 17, 2005

(54) Food supplement

(75) Inventor: Carl Franz de Vos Albrecht, Amanda Glen (ZA)

Correspondence Address: CROWELL & MORING LLP;INTELLECTUAL PROPERTY GROUP, P.O. BOX 14300, WASHINGTON, DC, 20044-4300, US

(73) Assignee: Guardian CIPLA MEDPRO (PTY) LTD, Bellville (ZA)

(21) Appl. No.: 10/513,106

(22) PCT Filed: May 2, 2003
(86) PCT No.: PCT/ZA03/00058

§ 371 Date: Jun. 1, 2005

(30) Foreign Application Priority Data
May 2, 2002 (ZA) 02/03470
Feb. 7, 2003 (ZA) 03/1055

(51) Int. Cl. A61K 31/525; A61K 35/78; A61K 31/015

(52) U.S. Cl. 424/725; 514/251; 514/763

(57) ABSTRACT

Food supplements are disclosed for supplementing the minerals, trace elements and phytochemicals in food. One form of the supplement comprises folic acid, lycopene and an extract of Aspalathus linearis (common name Red Bush) or Cyclopia intermedia (common name Honeybush) or a mixture of extracts of Aspalathus linearis and Cyclopia intermedia. Another form comprises lycopene and folic acid. The lycopene and folic acid are dispersed in canola oil.

### FIELD OF THE INVENTION

THIS INVENTION relates to a food supplement.

### BACKGROUND TO THE INVENTION

It has become evident during the past decade that the average diet of the vast majority of human beings is deficient in respect of certain specific vitamins, minerals, trace elements and phytochemicals.

There is growing evidence that many of these deficiencies could be involved in the initiation and progression of degenerative diseases, especially relating to cardiovascular disease and cancer which are the cause of up to 85% of mortality in many parts of the world.

Although there is a growing awareness of dietary deficiencies there is no consensus concerning the most important dietary components, which are deficient and the daily amounts to be used as supplements.

The total number of potentially deficient dietary components is in the order of about 10-20 and the required daily dosages cover a wide range.

The production of multivitamin/mineral/trace element supplements has become a large industry and the public have become used to taking daily tablets, capsules, syrups etc, as supplements.

If all potentially deficient dietary components were supplemented at maximum quantities the resulting product would be too bulky and expensive for general use.

The present invention seeks to provide novel food supplements.

### BRIEF DESCRIPTION OF THE INVENTION

According to a first aspect of the present invention there is provided a food supplement which comprises folic acid, lycopene and an extract of Aspalathus linearis (common name Red Bush) or Cyclopia intermedia (common name Honeybush) or a mixture of extracts of Aspalathus linearis and Cyclopia intermedia.

The Red Bush tea extract (as well as the Honeybush tea extract) is an aqueous extract produced by various processing steps, including filtration, stabilization, flocculation, membrane separation, concentration and drying.

The extract or mixture of extracts present should, at the recommended daily intake of the supplement, provide the following:

Total polyphenols—at least 15% and preferably at least 25% by weight (g Gallic Acid Equivalent/100 g)

Antioxidant capacity—at least 500 and preferably at least 1500 μmol Trolox Equivalent/g.

According to a second aspect of the present invention there is provided a food supplement which comprises lycopene and an extract of Aspalathus linearis or Cyclopia intermedia or a mixture of extracts of Aspalathus linearis and Cyclopia intermedia.

According to a third aspect of the present invention there is provided a food supplement which comprises folic acid and an extract of Aspalathus linearis or Cyclopia intermedia or a mixture of extracts of Aspalathus linearis and Cyclopia intermedia.

According to a fourth aspect of the present invention there is provided a food supplement including both lycopene and folic acid.

It is preferred that the defined constituents be in a pharmaceutically acceptable oil. The oil can be canola oil which is preferably extracted from the rape seeds by cold pressing. However, it is also possible for the constituents to be in the form of a powder, a syrup, tablets, a solution or a suspension. When constituents are in oil, the oil can be within a soft gel capsule or a hard shell capsule.

When folic acid is present it is preferably in a quantity that, at the recommended daily intake, gives a daily dosage of between 0.2 and 1.5 mg.

When lycopene is present it is preferably a quantity that, at the recommended daily intake of the supplement, gives a daily dosage of between 0.5 and 25 mg.

When said extract is present it is preferably in a quantity that, at the recommended daily intake of the supplement, gives a daily dosage of between 25 and 1500 mg. The extract preferably has a total polyphenols content of at least 15% (g Gallic Acid Equivalent/100 g) by weight and an antioxidant capacity of at least 500 μmol Trolox Equivalent/g.

The supplement can be provided in the form of soft gel capsules in which form the active ingredients are in a pharmaceutically acceptable oil. The oil used is preferably canola oil. Whilst one capsule can be formulated to give the requisite daily dosage, it is also possible to achieve the daily dosage by formulating the capsules so that two or more must be taken per day to achieve the required daily dosage.

A specific capsule (or capsules) in accordance with the present invention provides the following daily dosages:

200 to 750 mg of canola oil, between 0.4 and 1.0 mg of folic acid, between 4 and 10 mg of lycopene and between 100 and 500 mg of said extract.

According to a fifth aspect of the present invention there is provided a food supplement which provides the following daily dosages of the constituents specified,

| | | |
|---|---|---|
| folio acid | 0.2 to 1.5 | mg |
| vitamin B6 | 0.5 to 50 | mg |
| vitamin B12 | 0.01 to 0.03 | mg |
| methionine | 20 to 1000 | mg |
| choline | 2 to 50 | mg |
| lycopene | 0.5 to 25 | mg |
| vitamin C | 50 to 1000 | mg |
| vitamin E | 5 to 250 | mg |
| selenium | 0.02 to 0.25 | mg |
| extract | 25 to 1500 | mg |

the extract comprising Aspalathus linearis (common name Red Bush) or Cyclopia intermedia (common name Honeybush) or a mixture of extracts of Aspalathus linearis and Cyclopia intermedia.

The constituents listed are preferably dispersed in a pharmaceutically acceptable oil. Said oil is preferably canola oil and is present in an amount which, at the recommended daily intake of the supplement, gives a daily dosage of between 100 and 1000 mg. The oil is preferably recovered from the rape seeds by cold pressing.

According to a sixth aspect of the present invention there is provided a food supplement for combatting oxidation in the human body and which includes lycopene, vitamin C, vitamin E, selenium and said extract.

Said constituents are preferably present in quantities to give the following daily dosage:

| | | |
|---|---|---|
| lycopene | 0.5 to 25 | mg |
| vitamin C | 50 to 1000 | mg |
| vitamin E | 5 to 250 | mg |
| selenium | 0.02 to 0.25 | mg |
| extract | 25 to 1500 | mg |

According to a seventh aspect of the present invention there is provided a food supplement comprising lycopene dispersed in canola oil.

The quantity of lycopene is selected so that, at the recommended daily intake of the supplement, it gives a preferred daily dosage of between 0.5 to 25 mg.

1. A food supplement which comprises folic acid, lycopene and an extract of Aspalathus linearis (common name Red Bush) or Cyclopia Intermedia (common name Honeybush) or a mixture of extracts of Aspalathus linearis and Cyclopia intermedia.

2. A food supplement as claimed in claim 1, wherein the quantity of extract or mixture of extracts present provides, at the recommended daily intake of the supplement, the following:
Total polyphenois—at least 15% by weight (g Gallic Acid Equivalent/100 g)
Antioxidant capacity—at least 500 μmol Trolox Equivalent/g.

3. A food supplement as claimed in claim 2, wherein the extract or mixture of extracts present provides, at the recommended daily intake of the supplement, ast least 25% by weight (g Gallic Acid Equivalent/100 g).

4. A food supplement as claimed in claim 2 wherein the extract or mixture of extracts present provides, at the recommended daily intake of the supplement, at least 1500 μmol Trolox Equivalent/g.

5. A food supplement which comprises lycopene and an extract of Aspalathus linearis or Cyclopia intermedia or a mixture of extracts of Aspalathus linearis and Cyclopia intermedia.

6. A food supplement which comprises folic acid and an extract of Aspalathus linearis or Cyclopia intermedia or a mixture of extracts of Aspalathus linearis and Cyclopia intermedia.

7. A food supplement including both lycopene and folic acid.

8. A food supplement as claimed in claim 1, wherein the folic acid is present in a quantity that, at the recommended daily intake of the supplement, gives a daily dosage of between 0.2 and 1.5 mg.

9. A food supplement as claimed in claim 1, wherein the lycopene is present in a quantity that, at the recommended daily intake of the supplement, gives a daily dosage of between 0.5 and 25 mg.

10. A food supplement as claimed in claim 1, wherein said extract is present n a quantity that, at the recommended daily intake of the supplement, gives a daily dosage of between 25 and 1500 mg.

11. A food supplement as claimed in claim 1, and including a carrier in the form of a pharmaceutically acceptable oil in which the specified constituents are dispersed.

12. A food supplement as claimed in claim 11, wherein said oil is canola oil.

13. As a daily intake for supplementing the minerals, vitamins, trace elements and phytochemicals in food, one or more capsules providing the following daily dosages:
between 200 and 750 mg of canola oil, between 0.4 and 1.0 mg of folic acid, between 4 and 10 mg of lycopene and between 100 and 500 mg of an extract of Aspalathus linearis (common name Red Bush) or Cyclopia intermedia (common name Honeybush) or a mixture of extracts of Aspalathus linearis nd Cyclopia intermedia.

14. A food supplement which, at the recommended daily intake of the supplement, provides the following daily dosages of the constituents specified,

| | | |
|---|---|---|
| folic acid | 0.2 to 1.5 | mg |
| vitamin B6 | 0.5 to 50 | mg |
| vitamin B12 | 0.01 to 0.03 | mg |
| methionine | 20 to 1000 | mg |
| choline | 2 to 50 | mg |
| lycopene | 0.5 to 25 | mg |
| vitamin C | 50 to 1000 | mg |
| vitamin E | 5 to 250 | mg |
| selenium | 0.02 to 0.25 | mg |
| extract | 25 to 1500 | mg |

the extract comprising Aspalathus linearis (common name Red Bush) or Cyclopia intermedia (common name Honeybush) or a mixture of extracts of Aspalathus linearis and Cyclopia intermedia.

15. A food supplement as claimed in claim 14, wherein the constituents of the supplement are dispersed in a pharmaceutically acceptable oil.

16. A food supplement as claimed in claim 15, wherein the oil is canola oil and is present in an amount which, at the recommended daily intake of the supplement, gives a daily dosage of between 100 and 1000 mg.

17. A food supplement for combating oxidation in the human body and which includes lycopene, vitamin C, vitamin E, selenium and an extract of Aspalathus linearis (common name Red Bush) or Cyclopia intermedia (common name Honeybush) or a mixture of extracts of Aspalathus linearis and Cyclopia intermedia.

18. As a daily intake for supplementing the minerals, vitamins, trace elements and phytochemicals in food, a product to be taken by mouth which gives the following daily dosage:

| | | |
|---|---|---|
| lycopene | 0.5 to 25 | mg |
| vitamin C | 50 to 1000 | mg |
| vitamin E | 5 to 250 | mg |
| selenium | 0.02 to 0.25 | mg |
| extract | 25 to 1500 | mg |

the extract comprising Aspalathus linearis common name Red Bush) or Cyclopia intermedia (common name Honeybush) or a mixture of extracts of Aspalathus linearis and Cyclopia intermedia.

19. A food supplement comprising lycopene dispersed in canola oil.

20. A food supplement as claimed in claim 19, wherein the quantity of lycopene is selected so that, at the recommended daily intake of the supplement, it gives a daily dosage of between 0.5 to 25 mg.
